(19) United States
(12) Patent Application Publication
(10) Pub. No.: US 2009/0214601 A1
(43) Pub. Date: Aug. 27, 2009

(54) Porous Drug Delivery Devices and Related Methods

(76) Inventors: Ralph A. Chappa, Ham Lake, MN (US); James H. Arps, Chanhassen, MN (US); Steven J. Keough, St. Paul, MN (US)

Correspondence Address: PAULY, DEVRIES SMITH & DEFFNER, L.L.C., Plaza VII-Suite 3000, 45 South Seventh Street, MINNEAPOLIS, MN, 55402-1630, US

(21) Appl. No.: 12/238,641

(22) Filed: Sep. 26, 2008

Related U.S. Application Data
(60) Provisional application No. 60/976,035, filed on Sep. 28, 2007.

(51) Int. Cl. A61K 9/00; A61K 47/02; A61K 31/7036

(52) U.S. Cl. 424/400; 514/769; 514/40

(57) ABSTRACT

The invention relates to porous drug delivery devices and related methods. In an embodiment, the invention includes an active agent delivery system including a reservoir body defining a plurality of interconnected pores, an active agent disposed within the interconnected pores, and a first polymeric layer disposed over the reservoir body. In an embodiment, the invention includes an implantable medical device including a porous substrate defining a plurality of interconnected pores, an active agent disposed within the interconnected pores, and a first polymeric layer disposed over the reservoir body. In an embodiment, the invention includes a method of making an active agent delivery system including forming a porous reservoir body, inserting an active agent within the porous reservoir body, and applying a polymeric layer over the porous reservoir body. Other embodiments are also included herein.

This application claims the benefit of U.S. Provisional Application No. 60/976,035, filed Sep. 28, 2007, the contents of which are herein incorporated by reference.

### FIELD OF THE INVENTION

The invention relates to medical devices and methods. More specifically, the invention relates to porous drug delivery devices and related methods.

### BACKGROUND OF THE INVENTION

The administration of therapeutic agents (or active agents) is a cornerstone of modern medical care. Active agents can serve many purposes including preventing or treating infection, modulating the immune response of the patient, modulating tissue growth, etc.

Implantable medical devices are now commonly used to deliver active agents to tissues of the body. When delivered from an implantable medical device, active agents can be administered in a site-specific manner because the medical device can be positioned as desired within the body of a patient. Site specific administration can be advantageous because therapeutic effects on target tissues can be enhanced while side effects on other tissues can be decreased. In addition, some medical devices can enable the delivery of an active agent over an extended period of time in order to optimize therapeutic effect.

Delivery of an active agent from a medical device can be accomplished in various ways. For example, in one approach, the medical device can be directly loaded with the active agent. In another approach, an active agent eluting coating can be disposed over the medical device. In general, however, the delivery of active agents from medical devices in a controlled and predictable manner remains technically challenging. In addition, it can be difficult to obtain desirable elution profiles from many existing medical device drug delivery systems. Also, with existing systems it can be difficult to load as much active agent onto a medical device as is desired for some applications.

Therefore, a need still exists for devices and systems that can deliver active agents with desirable elution profiles and methods of making the same.

### SUMMARY OF THE INVENTION

The invention relates to porous drug delivery devices and related methods. In an embodiment, the invention includes an active agent delivery system including a reservoir body defining a plurality of interconnected pores; an active agent disposed within the interconnected pores; and a first polymeric layer disposed over the reservoir body.

In an embodiment, the invention includes an implantable medical device including a porous substrate defining a plurality of interconnected pores; an active agent disposed within the interconnected pores; and a first polymeric layer disposed over the reservoir body.

In an embodiment, the invention includes a method of making an active agent delivery system including forming a porous reservoir body; inserting an active agent within the porous reservoir body; and applying a polymeric layer over the porous reservoir body.

The above summary of the present invention is not intended to describe each discussed embodiment of the present invention. This is the purpose of the figures and the detailed description that follows.

### BRIEF DESCRIPTION OF THE DRAWINGS

The invention may be more completely understood in connection with the following drawings, in which:

FIG. 1 is a cross-sectional schematic view of an active agent delivery system in accordance with an embodiment of the invention.

FIG. 2 is a cross-sectional schematic view of an active agent delivery system in accordance with another embodiment of the invention.

FIG. 3 is a cross-sectional schematic view of an active agent delivery system in accordance with another embodiment of the invention.

FIG. 4 is a cross-sectional schematic view of an active agent delivery system in accordance with another embodiment of the invention.

FIG. 5 is a cross-sectional schematic view of an active agent delivery system in accordance with another embodiment of the invention.

FIG. 6 is a cross-sectional schematic view of an active agent delivery system in accordance with another embodiment of the invention.

FIG. 7 is a cross-sectional schematic view of an active agent delivery system in accordance with another embodiment of the invention.

FIG. 8 is a cross-sectional schematic view of an implantable medical device in accordance with another embodiment of the invention.

FIG. 9 is a perspective view of an implantable medical device in accordance with another embodiment of the invention.

FIG. 10 is a cross-sectional schematic view of an implantable medical device as taken along line 10-10′ of FIG. 9.

FIG. 11 is a cross-sectional schematic view of an implantable medical device in accordance with another embodiment of the invention.

FIG. 12 is graph contrasting zero-order active agent elution kinetics with first-order active agent elution kinetics.

FIG. 13 is a graph of active agent elution from a device as described in example 1 below.

FIG. 14 is a graph of active agent elution from a device as described in example 2 below.

While the invention is susceptible to various modifications and alternative forms, specifics thereof have been shown by way of example and drawings and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

One approach to administering an active agent from a medical device is to load the active agent on to the medical device, such as directly or with an active agent eluting coating, so that the active agent can elute from the medical device in vivo. However, it can be difficult to achieve desirable elution profiles with some types of active agent coating systems. In addition, it can be difficult to load as much active agent onto the medical device as is desired for some applications.

As demonstrated herein, porous reservoir materials can be used in combination with overlying elution control coatings in order to achieve desirable elution profiles. In addition, the use of porous reservoir materials can allow for the loading and delivery of relatively large amounts of active agents. In an embodiment, the invention includes an active agent delivery system including a reservoir body defining a plurality of interconnected pores; an active agent disposed within the interconnected pores; and a first polymeric layer disposed over the reservoir body.

Referring now to FIG. 1, a cross-sectional schematic view is shown of an active agent delivery system 100 in accordance with an embodiment of the invention. The active agent delivery system 100 includes a reservoir body 102 and a polymeric layer 106 (or top coat layer) disposed over the reservoir body 102. The reservoir body 102 can include many different materials, such as polymers, ceramics, metals, and the like. Exemplary reservoir body materials are described in greater detail below. The reservoir body 102 defines a plurality of interconnecting pores 104 (not to scale). The pores 104 can form an open-cell type structure.

An active agent (not shown) can be disposed within the pores 104. In some embodiments, two or more active agents can be disposed within the pores 104. The active agent can be configured to elute out of the pores 104 and through the polymeric layer 106 when the device is implanted in the body of a subject. Many different types of active agents can be used. Exemplary active agents include those described in greater detail below.

The reservoir body 102 can be of varying thickness depending on the particular application. By way of example, where it is important to maximize the total amount of active agent to be delivered, the reservoir body 102 can be relatively thick so that there is more room to hold active agent. In some embodiments, the reservoir body 102 can be 100 micrometers or more in thickness. In some embodiments, the reservoir body 102 can be 1 centimeter or more in thickness. The reservoir body 102 can take on various shapes. In some embodiments, the reservoir body 102 can be relatively flat and planar. In some embodiments, the reservoir body 102 can be polygonal. In some embodiments, the reservoir body 102 can be roughly spherical or toroidal. For example, in some embodiments, the reservoir body 102 can take on the shape of a bead.

The polymeric layer 106 (or top coat layer) can include polymers used to control the elution rate of active agents eluting out from the pores 104. Exemplary polymers of the polymeric layer 106 are described in greater detail below. The thickness of the polymeric layer can vary depending on the particular polymer used and the desired effect on the elution rate of the active agent. In some embodiments, the polymeric layer can be about 10 nanometers or more in thickness. In some embodiments, the polymeric layer can be about 0.1 micrometers or more in thickness. In some embodiments, the polymeric layer can be about 10 micrometers or more in thickness. In some applications, if the polymeric layer is too thick, the resulting elution rate may be undesirably slow. In some embodiments, the polymeric layer is about 500 micrometers or less in thickness. In some embodiments, the polymeric layer is about 300 micrometers or less in thickness. In some embodiments, the polymeric layer is about 100 micrometers or less in thickness.

The term “porosity” as used herein shall refer to the specific percentage of volume within an object that is taken up by pores. By way of example, if a sphere has a radius of 3.5 millimeters (and therefore a volume of 179.15 mm³), then if it has a porosity of 35% the pores take up a total volume of approximately 62.7 mm. While not intending to be bound by theory, the porosity of the reservoir body 102 can impact aspects of the active agent delivery system 100, including its construction and its performance during use. The porosity of the reservoir body 102 should be sufficiently high so that the reservoir body 102 can carry as much active agent as desired. In addition, in some embodiments, the porosity of the reservoir body 102 should be sufficiently high so that the pores are interconnected, forming an open-cell structure. This can be significant, particularly where the reservoir body 102 is made of a material that is impermeable to the migration of the active agent. For example, in some embodiments, the reservoir body 102 can be made of high density polyethylene (HDPE). Generally, active agents can pass through pores in HDPE, but cannot pass directly through the HDPE itself. In some embodiments, the porosity of the reservoir body 102 should be at least about 5 percent. In some embodiments, the porosity of the reservoir body 102 should be at least about 30 percent.

If the porosity of the reservoir body 102 is too high, various aspects of the delivery system 100 can be affected. For example, in some applications, it may be desirable for the reservoir body to maintain a degree of structural integrity. Structural integrity of some reservoir body 102 materials may be reduced if the porosity is above a threshold level. In some embodiments, the porosity of the reservoir body 102 is less than about 90 percent. In some embodiments, the porosity of the reservoir body 102 is less than about 50 percent. In some embodiments, the porosity of the reservoir body 102 is between about 5 percent and about 90 percent. In some embodiments, the porosity of the reservoir body 102 is between about 30 percent and about 50 percent.

The size of individual pores 104 within the reservoir body 102 can also affect aspects of the active agent delivery system 100. While not intending to be bound by theory, it is believed that, for example, the size of individual pores 104 can change the capillary effect exhibited by fluids within the pores. The capillary effect of fluids within the pores can be significant because it can function to draw in fluids, such as an active agent solution during construction of the active agent delivery system, or draw in a bodily fluid that can solvate the active agent resulting in elution in vivo. In practice, the average pore size can be influenced by many factors including the method of making the reservoir porous and the chemical properties of the reservoir material. In some embodiments, the average pore size should be big enough so that the active agent can be disposed within the pores at a desired loading level. In some embodiments, the average pore size is greater than about 0.1 micrometers. In some embodiments, the average pore size is greater than about 0.5 micrometers. For some applications, the average pore size should be small enough so as to exhibit a desirable capillary effect. In some embodiments, the average pore size is less than about 50 micrometers. In some embodiments, the average pore size is less than about 20 micrometers. In some embodiments, the average pore size is between about 0.1 micrometers and about 50 micrometers. In some embodiments, the average pore size is between about 0.5 micrometers and about 20 micrometers.

The pores 104 in the reservoir body 102 are defined by pore surfaces 114. The pore surfaces 114 generally derive their functional properties from the material used to make the reservoir body 102. For example, in some embodiments, the material used to make the reservoir body 102 includes a polymer, many of which are relatively non-polar, and as a result the pore surfaces 114 can exhibit non-polar characteristics. The functional properties of the pore surfaces 114, such as polar or non-polar nature, can be significant because the pore surfaces 114 can interact with the active agent disposed within the pores. The interaction between the pore surfaces 114 and the active agent can affect the process of inserting the active agent into the pores as well as affecting the release characteristics of active agents migrating out of the pores. As a specific example, in some embodiments, the pore surfaces 114 can have a relatively polar surface, such as because of the presence of a charged species on the pore surface 114. Where the pore surface has a negative charge, for example, and the active agent has a positive charge, they may interact strongly and the resulting active agent elution rate may be relatively slow. Based on this specific example, it will be appreciated that the net result of the pore surface characteristics can depend on both the pore surfaces 114 themselves and the specific active agent to be disposed therein. In some embodiments, the pore surfaces 114 can be non-polar. In some embodiments, the pore surfaces 114 can be polar. In some embodiments the pore surfaces 114 can have a positive charge. In some embodiments, the pore surfaces 114 can have a negative charge. In some embodiments, the pore surfaces 114 can be charge neutral.

In some embodiments, the characteristic of the pore surfaces 114 can be manipulated independently of the material(s) used to make the reservoir body 102. For example, surface property modifying agents, such as surfactants, can be used in order to render the pore surfaces 114 relatively polar or non-polar depending on what is desired for the particular application.

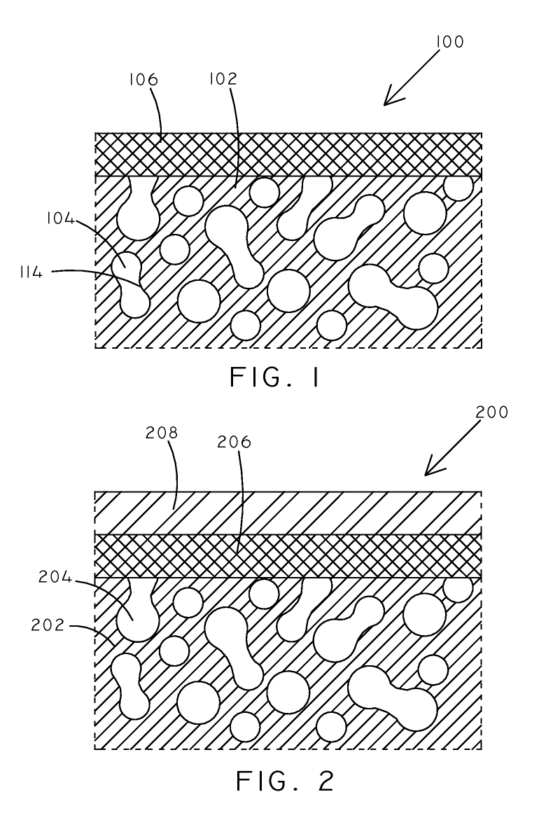

It will be appreciated that many different configurations of active agent delivery systems are included within embodiments herein. Referring now to FIG. 2, a schematic view of an active agent delivery system 200 is shown with multiple polymeric layers (or multiple top coat layers). The active agent delivery system 200 includes a reservoir body 202 defining a plurality of interconnecting pores 204. The pores 204 can form an open-cell type structure. An active agent (not shown) can be disposed within the pores 204. A first polymeric layer 206 (or top coat layer) is disposed over the reservoir body 202. A second polymeric layer 208 (or top coat layer) is disposed over the first polymeric layer 206. Both the first polymeric layer 206 and the second polymeric layer 208 can include polymeric layer materials, such as those described in more detail below. The first polymeric layer 206 and the second polymeric layer 208 can include either the same polymers or different polymers. For example, in an embodiment, the first polymer layer 206 includes a polyalkylmethacrylate and the second polymer layer 208 includes a parylene.

In some embodiments, either one or both of the first polymer layer 206 and the second polymer layer 208 can include an active agent, such as those described in more detail below. The active agents of the first polymer layer 206 and the second polymer layer 208 can be the same as one another or different. The active agents of the first polymer layer 206 and the second polymer layer 208 can be the same active agent as in the pores 204 of the reservoir body 202 or can be different active agents. In some embodiments, either one or both of the first polymer layer 206 and the second polymer layer 208 contains an active agent that is configured to elute at a rate different from the active agent within the pores 204 of the reservoir body 202.

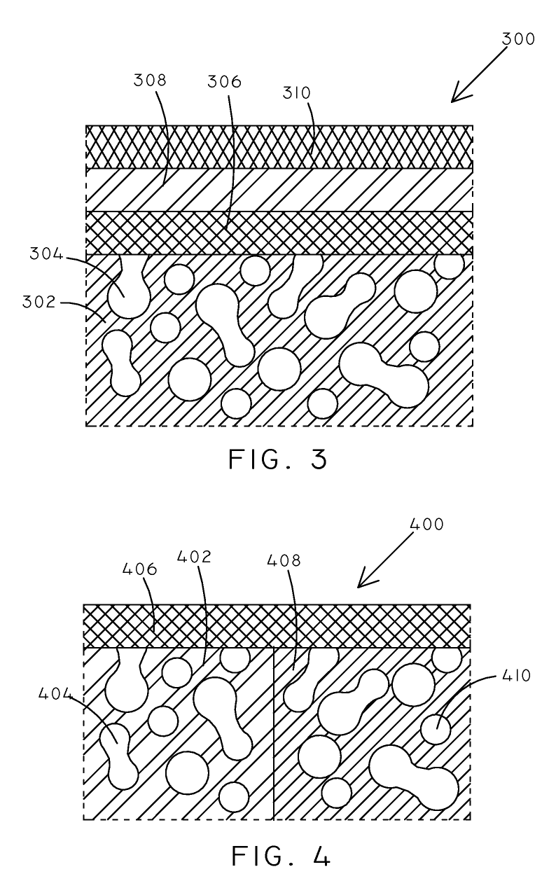

Referring now to FIG. 3, a schematic view of an active agent delivery system 300 is shown with multiple polymeric layers. The active agent delivery system 300 includes a reservoir body 302 defining a plurality of interconnecting pores 304. The pores 304 can form an open-cell type structure. An active agent (not shown) can be disposed within the pores 304. A first polymeric layer 306 (or top coat layer) is disposed over the reservoir body 302. A second polymeric layer 308 (or top coat layer) is disposed over the first polymeric layer 306. A third polymeric layer 310 (or top coat layer) is disposed over the second polymeric layer 308. The first polymeric layer 306, the second polymeric layer 308, and the third polymeric layer 310 can include polymeric layer materials, such as those described in more detail below. The first polymeric layer 306, the second polymeric layer 308, and the third polymeric layer 310 can include either the same polymers as one another or different polymers. Each of the first polymeric layer 306, the second polymeric layer 308, and the third polymeric layer 310 can also include one or more active agents.

Referring now to FIG. 4, a schematic view of an active agent delivery system 400 is shown including a first reservoir body 402 defining a first plurality of interconnecting pores 404. The active agent delivery system 400 also includes a second reservoir body 408 defining a second plurality of interconnecting pores 410. In some embodiments, the first plurality of interconnecting pores 404 are in fluid communication with the second plurality of interconnecting pores 410. However, in other embodiments, the first plurality of interconnecting pores 404 are not in fluid communication with the second plurality of interconnecting pores 410. The first reservoir body 402 and the second reservoir body 408 can include materials such as those described in greater detail below. The first reservoir body 402 can include the same material as the second reservoir body 408 or can include different material(s).

An active agent (not shown) can be disposed within the first plurality of pores 404. An active agent (not shown) can also be disposed within the second plurality of pores 410. The active agent in the first plurality of pores 404 can be either the same or different than the active agent in the second plurality of pores 410. Exemplary active agents are described in greater detail below. Active agents in the first plurality of pores 404 can be configured to elute at a rate the same or different than active agent in the second plurality of pores 410. A polymeric layer 406 can be disposed over both the first reservoir body 402 and the second reservoir body 408.

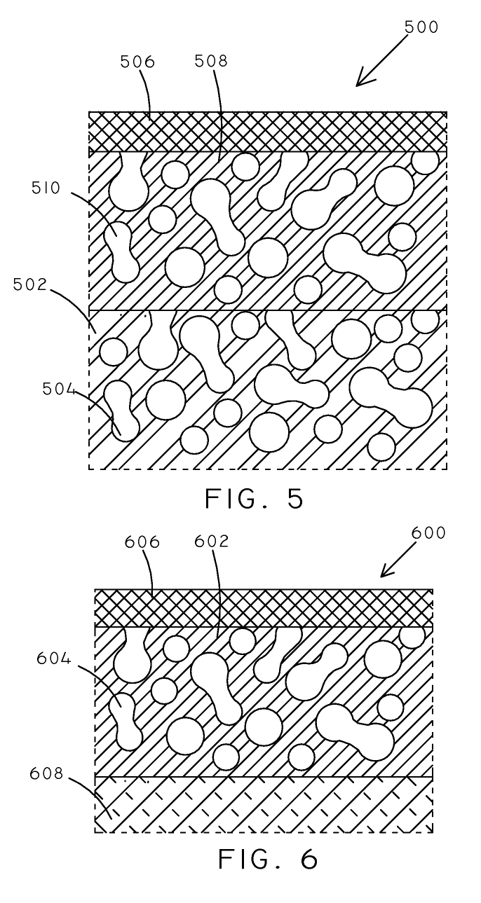

Referring now to FIG. 5, a schematic view of an active agent delivery system 500 is shown including a first reservoir body 502 defining a first plurality of interconnecting pores 504. The active agent delivery system 500 also includes a second reservoir body 508 defining a second plurality of interconnecting pores 510. The second reservoir body 508 is disposed on top of the first reservoir body 502. The first plurality of interconnecting pores 504 can be in fluid communication with the second plurality of interconnecting pores 510. The first reservoir body 502 and the second reservoir body 508 can include materials such as those described in greater detail below. The first reservoir body 502 can include the same material as the second reservoir body 508 or can include different materials.

An active agent (not shown) can be disposed within the first plurality of pores 504. An active agent (not shown) can also be disposed within the second plurality of pores 510. The active agent in the first plurality of pores 504 can be either the same or different than the active agent in the second plurality of pores 510. Exemplary active agents are described in greater detail below. Active agents in the first plurality of pores 504 can be configured to elute at a rate the same or different than the active agent in the second plurality of pores 510. A polymeric layer 506 can be disposed over both the first reservoir body 502 and the second reservoir body 508.

In some embodiments, the active agent delivery system can include an underlying support layer for purposes of structural integrity, manufacturing ease, or design preference. Referring now to FIG. 6, a cross-sectional schematic view is shown of an active agent delivery system 600 in accordance with another embodiment of the invention. The active agent delivery system 600 includes a support layer 608. The support layer 608 can be a polymer, a ceramic, a metal, or the like. The support layer 608 can be either porous or non-porous. A reservoir body 602 can be disposed over the support layer 608, and a polymeric layer 606 disposed over the reservoir body 602. The reservoir body 602 can include many different materials, such as polymers, ceramics, and/or metals, as described in greater detail below. The reservoir body 602 defines a plurality of interconnecting pores 604 (not to scale). The pores 604 form an open-cell type structure. An active agent (not shown) can be disposed within the pores 604.

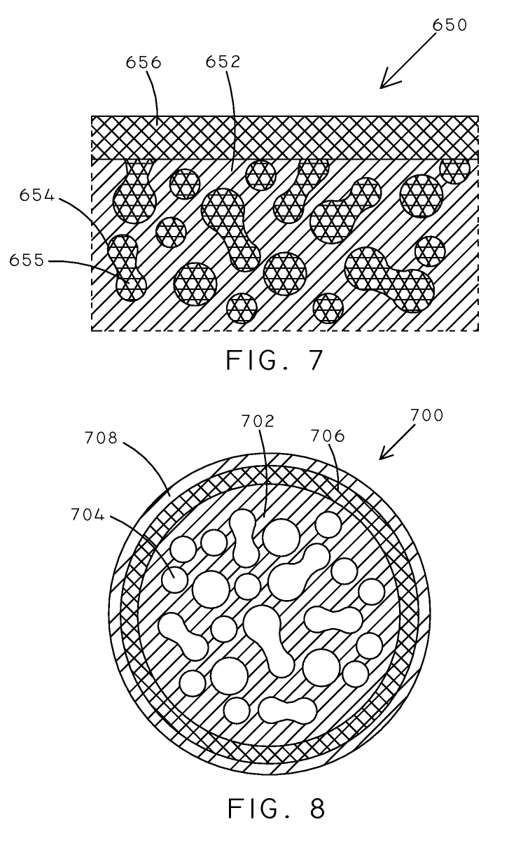

In some embodiments, an active agent can be mixed with a polymer composition and the combination can then be disposed within pores of the reservoir body of an active agent delivery system. Referring now to FIG. 7, a cross-sectional schematic view is shown of an active agent delivery system 650 in accordance with an embodiment of the invention. The active agent delivery system 650 includes a reservoir body 652 defining a plurality of interconnecting pores 654. The pores 654 can form an open-cell type structure. A composition 655 comprising an active agent and a polymer can be disposed within the pores 654. The polymer of the composition can include those described herein with respect to polymeric layers. In some embodiments, the polymer of the composition can be a degradable polymer. In embodiment, the polymer of the composition can include a polyalkylmethacrylate. In an embodiment, the polymer of the composition can include a degradable polymer. One or more polymeric layers 656 (or top coat layers) can be disposed over the reservoir body 652.

Embodiments of the invention can also include implantable medical devices configured to elute an active agent. Referring now to FIG. 8, a cross-sectional schematic view is shown of an implantable medical device 700 including a porous substrate 702 (or reservoir) defining a plurality of interconnected pores 704. The implantable medical device 700 can be, for example, a bead. The pores 704 (not to scale) form an open-cell type structure. The porous substrate 702 can include many different materials, such as polymers, ceramics, and/or metals, as described in greater detail below. A first polymeric layer 706 can be disposed over the porous substrate 702. A second polymeric layer 708 can be disposed over the first polymeric layer 706. The first polymeric layer 706 and the second polymeric layer 708 can include various polymers, such as the exemplary polymers described in greater detail below. In a particular embodiment, the first polymeric layer 706 includes a polyalkylmethacrylate and the second polymeric layer 708 includes a parylene. An active agent (not shown) can be disposed within the pores 704.

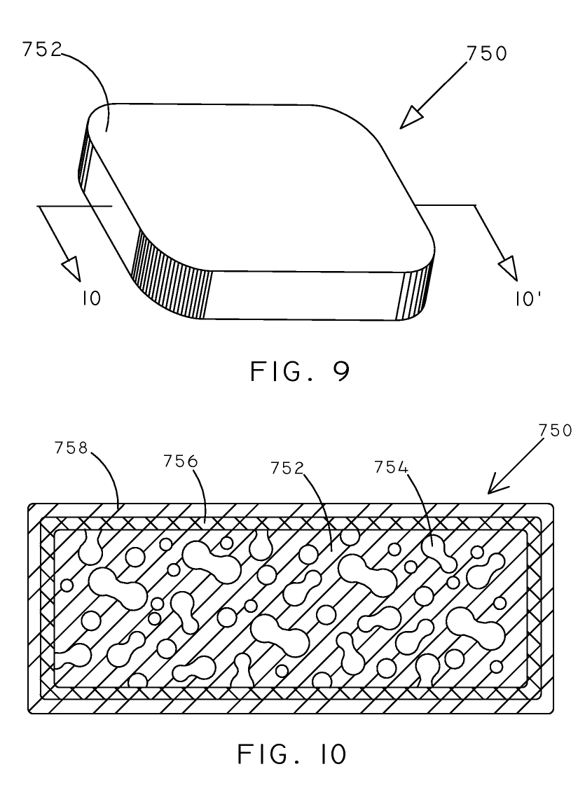

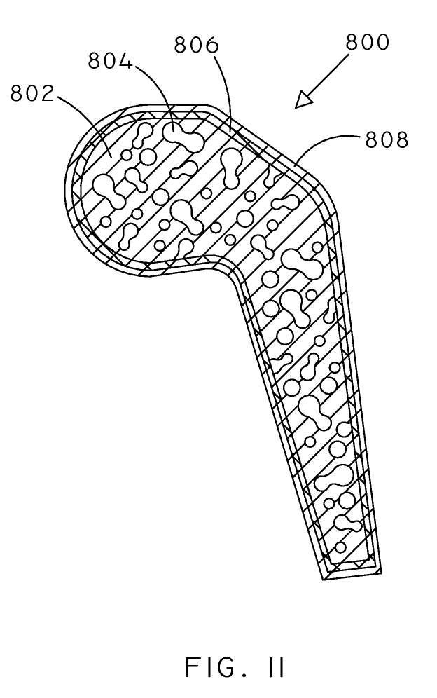

In some embodiments, implantable medical devices of the invention can include orthopedic devices. Referring now to FIG. 9, an embodiment of a spacer block 750 is shown in accordance with an embodiment of the invention. Spacer blocks are frequently used in knee revision surgeries. The spacer block can be configured to elute active agents, such as antibiotics, over a period of time. FIG. 10 is a cross-sectional view (not to scale) of the implantable spacer block 750 taken along line 10-10′ of FIG. 9. The spacer block can include a reservoir body 752 defining a plurality of interconnecting pores 754. The pores 754 can form an open-cell type structure. In some embodiments, the reservoir body comprises a polymer. In some embodiments, the reservoir body comprises a ceramic.

An active agent (not shown) can be disposed within the pores 754. A first polymeric layer 756 (or top coat layer) is disposed over the reservoir body 752. A second polymeric layer 758 (or top coat layer) is disposed over the first polymeric layer 756. Both the first polymeric layer 756 and the second polymeric layer 758 can include polymeric layer materials, such as those described in more detail below. The first polymeric layer 756 and the second polymeric layer 758 can include either the same polymers or different polymers. For example, in an embodiment, the first polymer layer 756 includes a polyalkylmethacrylate and the second polymer layer 758 includes a parylene. In some embodiments, the first and second polymer layers are disposed over only a portion of the reservoir body. For example, in some embodiments, the first and second polymer layers are only disposed over portions of the reservoir body not exposed to substantial friction in vivo.

Referring now to FIG. 11, a cross-sectional view of implantable medical device 800 is shown in accordance with another embodiment of the invention. In this embodiment, the implantable medical device 800 is a femoral portion of an artificial hip joint. The implantable medical device 800 includes a reservoir body 802 defining a plurality of interconnecting pores 804. The pores 804 can form an open-cell type structure. In some embodiments, the reservoir body 802 comprises a polymer. In some embodiments, the reservoir body 802 comprises a ceramic. An active agent (not shown) can be disposed within the pores 804. A first polymeric layer 806 (or top coat layer) is disposed over the reservoir body 802. A second polymeric layer 808 (or top coat layer) is disposed over the first polymeric layer 806. Both the first polymeric layer 806 and the second polymeric layer 808 can include polymeric layer materials, such as those described in more detail below. The first polymeric layer 806 and the second polymeric layer 808 can include either the same polymers or different polymers. For example, in an embodiment, the first polymer layer 806 includes a polyalkylmethacrylate and the second polymer layer 808 includes a parylene.

### Elution Kinetics

Many active agent elution control coatings exhibit kinetics characterized by an initial burst followed by a rapid decline in the release rate. This type of pattern is sometimes referred to as first-order release kinetics. However, in some circumstances it can be desirable to release active agents in a steady fashion wherein the active agent release rate is relatively constant over an extended period of time. This type of pattern is sometimes referred to as zero-order release kinetics. Therapeutic effects can be enhanced in some instances by zero-order release kinetics. This is because zero-order release kinetics can facilitate maintaining a therapeutic concentration of the active agent in target tissues over an extended period of time.

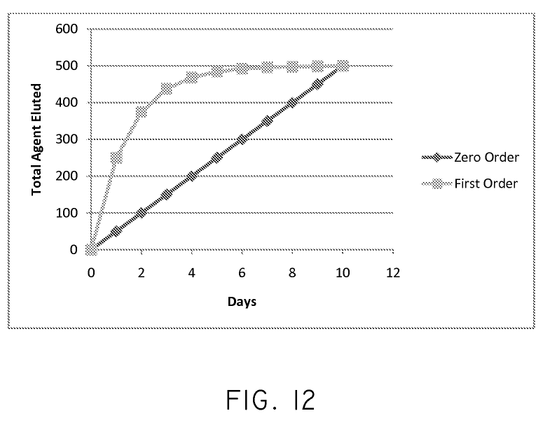

Referring now to FIG. 12, a graph is shown exhibiting idealized plots of release profiles consistent with both zero-order kinetics and first-order release kinetics. As can be seen, the idealized plot of first-order kinetics exhibits a relatively large initial release rate (burst) followed by a rapid reduction in the release rate as the total amount of active agent released increases. In contrast, the zero-order plot shows a constant active-agent release rate that continues until the active agent has been completely eluted off.

Embodiments described herein can be configured to elute active agent with various elution profiles including first-order release kinetics and zero-order release kinetics. In some embodiments, first-order or zero-order active agent release kinetics can be achieved over a significant period of time. For example, some embodiments of systems and devices herein can be configured to provide active agent release over a period of time of ten days or more. Some embodiments of systems and devices herein can be configured to provide active agent release over a period of time of twenty days or more. Some embodiments of systems and devices herein can be configured to provide active agent release over a period of time of thirty days or more. Some embodiments of systems and devices herein can be configured to provide active agent release over a period of time of sixty days or more. Some embodiments of systems and devices herein can be configured to provide active agent release over a period of time of ninety days or more.

In some embodiments, system and device herein can be configured to elute an amount of the active agent between 30 and 60 days that is at least equal to 90% of the amount eluted between 0 days and 30 days. In some embodiments, system and device herein can be configured to elute an amount of the active agent between 30 and 60 days that is at least equal to 80% of the amount eluted between 0 days and 30 days. In some embodiments, the system can be configured to elute at least about 20% of the total amount of the active agent after being disposed in vivo for at least 60 days.

### Porous Reservoir Materials

Porous reservoirs of embodiments described herein can include various materials such as polymers, ceramics, metals, and the like. In some embodiments, the porous reservoir can include a ceramic. Exemplary ceramics can include alumina, hydroxyapatite, calcium phosphate, calcium triphosphate, pyrolytic carbon, sapphire, silica, silicon carbide, silicon nitride, zirconia, and the like. In some embodiments, the porous reservoir can include a polymer. Exemplary polymers can include polyolefins such as polyethylene (including high-density polyethylene (HDPE) and ultra high molecular weight polyethylene (UHMWPE)), polypropylene, polyamides (such as NYLON), polysiloxanes, polyurethanes, polyethers, polyesters, polyalkylacrylates, epoxy resins, and the like. In some embodiments, the polymer can include one with a Shore durometer hardness of at least about 50D. However, in some embodiments, the polymer can be substantially flexible. In some embodiments the porous reservoir can include a flexible foam material. In some embodiments, the porous reservoir can include a metal. Exemplary metals can include titanium, titanium alloys, stainless steel (an alloy including iron, chrome, and nickel), cobalt-chrome alloys, and the like. In some embodiments, the porous reservoir includes a material with a Rockwell hardness of greater than about HRC 40. In some embodiments, the porous reservoir can include naturally derived materials such as bone and/or cartilage.

Many different techniques can be used to render ceramics, polymers, metals, and materials porous. For example, phase extraction techniques can be used to render various types of material porous. Other techniques can include sintering, molding, casting, sol-gel techniques, aerogel techniques, spraying techniques, and the like.

In some applications, it can be desirable for the porous reservoir to exhibit a degree of structural rigidity. In some embodiments, the porous reservoir includes a material with a shear modulus of greater than about 3 GPa.

### Polymeric Layer Materials

Polymeric layers for enhancing elution control can include one or more polymers. In an embodiment, the polymeric layer includes a plurality of polymers, including a first polymer and a second polymer. When the polymeric layer contains only one polymer, it can be either a first or second polymer as described herein. As used herein, the term “(meth)acrylate” when used in describing polymers shall include such molecules in the acrylic and/or methacrylic form (corresponding to the acrylates and/or methacrylates, respectively). Polymers of the polymeric layer can be degradable or non-degradable. In some embodiments, the polymers of the polymeric layer are non-degradable.

Examples of suitable first polymers include poly(alkyl(meth)acrylates), and in particular, those with alkyl chain lengths from 2 to 8 carbons, and with molecular weights from 50 kilodaltons to 900 kilodaltons. An exemplary first polymer is poly(n-butyl methacrylate) (pBMA). Such polymers are available commercially, e.g., from Aldrich, with molecular weights ranging from about 200,000 Daltons to about 320,000 Daltons, and with varying inherent viscosity, solubility, and form (e.g., as crystals or powder).

Examples of suitable first polymers also include polymers selected from the group consisting of poly(aryl(meth)acrylates), poly(aralkyl(meth)acrylates), and poly(aryloxyalkyl(meth)acrylates). Such terms are used to describe polymeric structures wherein at least one carbon chain and at least one aromatic ring are combined with acrylic groups, typically esters, to provide a composition. In particular, exemplary polymeric structures include those with aryl groups having from 6 to 16 carbon atoms and with weight average molecular weights from about 50 to about 900 kilodaltons. Suitable poly(aralkyl(meth)acrylates), poly(arylalky(meth)acrylates) or poly(aryloxyalkyl (meth)acrylates) can be made from aromatic esters derived from alcohols also containing aromatic moieties. Examples of poly(aryl(meth)acrylates) include poly(9-anthracenyl methacrylate), poly(chlorophenylacrylate), poly(methacryloxy-2-hydroxybenzophenone), poly(methacryloxybenzotriazole), poly(naphthylacrylate) and -methacrylate), poly(4-nitrophenyl acrylate), poly(pentachloro(bromo, fluoro) acrylate) and -methacrylate), and poly(phenyl acrylate) and -methacrylate). Examples of poly(aralkyl(meth)acrylates) include poly(benzyl acrylate) and -methacrylate), poly(2-phenethyl acrylate) and -methacrylate), and poly(1-pyrenylmethyl methacrylate). Examples of poly(aryloxyalkyl (meth)acrylates) include poly(phenoxyethyl acrylate) and -methacrylate), and poly(polyethylene glycol phenyl ether acrylates) and -methacrylates) with varying polyethylene glycol molecular weights.

Examples of suitable second polymers are available commercially and include poly(ethylene-co-vinyl acetate) (pEVA) having vinyl acetate concentrations of between about 10% and about 50%, in the form of beads, pellets, granules, etc. pEVA co-polymers with lower percent vinyl acetate become increasingly insoluble in typical solvents, whereas those with higher percent vinyl acetate become decreasingly durable.

An exemplary polymer mixture for use herein includes mixtures of pBMA and pEVA. This mixture of polymers can be used with absolute polymer concentrations (i.e., the total combined concentrations of both polymers in the coating material), of between about 0.25 wt. % and about 99 wt. %. This mixture can also be used with individual polymer concentrations in the coating composition of between about 0.05 wt. % and about 99 wt. %. In one embodiment the polymer mixture includes pBMA with a molecular weight of from 100 kilodaltons to 900 kilodaltons and a pEVA copolymer with a vinyl acetate content of from 24 to 36 weight percent. In an embodiment the polymer mixture includes pBMA with a molecular weight of from 200 kilodaltons to 400 kilodaltons and a pEVA copolymer with a vinyl acetate content of from 24 to 36 weight percent. The concentration of the active agent or agents dissolved or suspended in the coating mixture can range from 0.01 to 99 percent, by weight, based on the weight of the final coating material.

Second polymers of the invention can also comprise one or more polymers selected from the group consisting of (i) poly(alkylene-co-alkyl(meth)acrylates, (ii) ethylene copolymers with other alkylenes, (iii) polybutenes, (iv) diolefin derived non-aromatic polymers and copolymers, (v) aromatic group-containing copolymers, and (vi) epichlorohydrin-containing polymers. First polymers of the invention can also comprise a polymer selected from the group consisting of poly(alkyl(meth)acrylates) and poly(aromatic (meth)acrylates), where “(meth)” will be understood by those skilled in the art to include such molecules in either the acrylic and/or methacrylic form (corresponding to the acrylates and/or methacrylates, respectively).

Poly(alkylene-co-alkyl(meth)acrylates) include those copolymers in which the alkyl groups are either linear or branched, and substituted or unsubstituted with non-interfering groups or atoms. Such alkyl groups can comprise from 1 to 8 carbon atoms, inclusive. Such alkyl groups can comprise from 1 to 4 carbon atoms, inclusive. In an embodiment, the alkyl group is methyl. In some embodiments, copolymers that include such alkyl groups can comprise from about 15% to about 80% (wt) of alkyl acrylate. When the alkyl group is methyl, the polymer contains from about 20% to about 40% methyl acrylate in some embodiments, and from about 25% to about 30% methyl acrylate in a particular embodiment. When the alkyl group is ethyl, the polymer contains from about 15% to about 40% ethyl acrylate in an embodiment, and when the alkyl group is butyl, the polymer contains from about 20% to about 40% butyl acrylate in an embodiment.

Alternatively, second polymers for use in this invention can comprise ethylene copolymers with other alkylenes, which in turn, can include straight and branched alkylenes, as well as substituted or unsubstituted alkylenes. Examples include copolymers prepared from alkylenes that comprise from 3 to 8 branched or linear carbon atoms, inclusive. In an embodiment, copolymers prepared from alkylene groups that comprise from 3 to 4 branched or linear carbon atoms, inclusive. In a particular embodiment, copolymers prepared from alkylene groups containing 3 carbon atoms (e.g., propene). By way of example, the other alkylene is a straight chain alkylene (e.g., 1-alkylene). Exemplary copolymers of this type can comprise from about 20% to about 90% (based on moles) of ethylene. In an embodiment, copolymers of this type comprise from about 35% to about 80% (mole) of ethylene. Such copolymers will have a molecular weight of between about 30 kilodaltons to about 500 kilodaltons. Exemplary copolymers are selected from the group consisting of poly(ethylene-co-propylene), poly(ethylene-co-1-butene), polyethylene-co-1-butene-co-1-hexene) and/or poly(ethylene-co-1-octene).

“Polybutenes” suitable for use in the present invention include polymers derived by homopolymerizing or randomly interpolymerizing isobutylene, 1-butene and/or 2-butene. The polybutene can be a homopolymer of any of the isomers or it can be a copolymer or a terpolymer of any of the monomers in any ratio. In an embodiment, the polybutene contains at least about 90% (wt) of isobutylene or 1-butene. In a particular embodiment, the polybutene contains at least about 90% (wt) of isobutylene. The polybutene may contain non-interfering amounts of other ingredients or additives, for example it can contain up to 1000 ppm of an antioxidant (e.g., 2,6-di-tert-butyl-methylphenol). By way of example, the polybutene can have a molecular weight between about 150 kilodaltons and about 1,000 kilodaltons. In an embodiment, the polybutene can have between about 200 kilodaltons and about 600 kilodaltons. In a particular embodiment, the polybutene can have between about 350 kilodaltons and about 500 kilodaltons. Polybutenes having a molecular weight greater than about 600 kilodaltons, including greater than 1,000 kilodaltons are available but are expected to be more difficult to work with.

Additional alternative second polymers include diolefin-derived, non-aromatic polymers and copolymers, including those in which the diolefin monomer used to prepare the polymer or copolymer is selected from butadiene (CH₂═CH—CH═CH₂) and/or isoprene (CH₂═C(CH₃)CH═CH₂). In an embodiment, the polymer is a homopolymer derived from diolefin monomers or is a copolymer of diolefin monomer with non-aromatic mono-olefin monomer, and optionally, the homopolymer or copolymer can be partially hydrogenated. Such polymers can be selected from the group consisting of polybutadienes prepared by the polymerization of cis-, trans- and/or 1,2-monomer units, or from a mixture of all three monomers, and polyisoprenes prepared by the polymerization of cis-1,4- and/or trans-1,4-monomer units. Alternatively, the polymer is a copolymer, including graft copolymers, and random copolymers based on a non-aromatic mono-olefin monomer such as acrylonitrile, and an alkyl(meth)acrylate and/or isobutylene. In an embodiment, when the mono-olefin monomer is acrylonitrile, the interpolymerized acrylonitrile is present at up to about 50% by weight; and when the mono-olefin monomer is isobutylene, the diolefin is isoprene (e.g., to form what is commercially known as a “butyl rubber”). Exemplary polymers and copolymers have a molecular weight between about 150 kilodaltons and about 1,000 kilodaltons. In an embodiment, polymers and copolymers have a molecular weight between about 200 kilodaltons and about 600 kilodaltons.

Additional alternative second polymers include aromatic group-containing copolymers, including random copolymers, block copolymers and graft copolymers. In an embodiment, the aromatic group is incorporated into the copolymer via the polymerization of styrene. In a particular embodiment, the random copolymer is a copolymer derived from copolymerization of styrene monomer and one or more monomers selected from butadiene, isoprene, acrylonitrile, a C₁-C₄ alkyl(meth)acrylate (e.g., methyl methacrylate) and/or butene. Useful block copolymers include copolymer containing (a) blocks of polystyrene, (b) blocks of an polyolefin selected from polybutadiene, polyisoprene and/or polybutene (e.g., isobutylene), and (c) optionally a third monomer (e.g., ethylene) copolymerized in the polyolefin block. The aromatic group-containing copolymers contain about 10% to about 50% (wt.) of polymerized aromatic monomer and the molecular weight of the copolymer is from about 300 kilodaltons to about 500 kilodaltons. In an embodiment, the molecular weight of the copolymer is from about 100 kilodaltons to about 300 kilodaltons.

Additional alternative second polymers include epichlorohydrin homopolymers and poly(epichlorohydrin-co-alkylene oxide) copolymers. In an embodiment, in the case of the copolymer, the copolymerized alkylene oxide is ethylene oxide. By way of example, epichlorohydrin content of the epichlorohydrin-containing polymer is from about 30% to 100% (wt). In an embodiment, epichlorohydrin content is from about 50% to 100% (wt). In an embodiment, the epichlorohydrin-containing polymers have a molecular weight from about 100 kilodaltons to about 300 kilodaltons.

Polymers used in embodiments of the invention can also include those described in U.S. patent application Ser. No. 11/493,346, entitled “DEVICES, ARTICLES, COATINGS, AND METHODS FOR CONTROLLED ACTIVE AGENT RELEASE OR HEMOCOMPATIBILITY”, the contents of which is herein incorporated by reference. As a specific example, polymers can include random copolymers of butyl methacrylate-co-acrylamido-methyl-propane sulfonate (BMA-AMPS). In some embodiments, the random copolymer can include AMPS in an amount equal to about 0.5 mol. % to about 40 mol. %.

Polymeric layers included with embodiments of the invention can include degradable polymers. The term “degradable” as used herein with reference to polymers, shall refer to those natural or synthetic polymers that break down under physiological conditions into constituent components over a period of time. By way of example, many degradable polymers include hydrolytically unstable linkages in the polymeric backbone. The cleavage of these unstable linkages leads to degradation of the polymer. The terms “erodible”, “bioerodible”, “biodegradable” and “non-durable” shall be used herein interchangeably with the term “degradable”.

Degradable (or biodegradable) polymers can include both synthetic and natural polymers. Synthetic degradable polymers can include: degradable polyesters (such as poly(glycolic acid), poly(lactic acid), poly(lactic-co-glycolic acid), poly(dioxanone), polylactones (e.g., poly(caprolactone)), poly(3-hydroxybutyrate), poly(3-hydroxyvalerate), poly(valerolactone), poly(tartronic acid), poly(B-malonic acid), poly(propylene fumarate)); degradable polyesteramides; degradable polyanhydrides (such as poly(sebacic acid), poly(1,6-bis(carboxyphenoxy)hexane, poly(1,3-bis(carboxyphenoxy)propane); degradable polycarbonates; degradable polyiminocarbonates; degradable polyarylates; degradable polyorthoesters; degradable polyurethanes; degradable polyphosphazenes; and degradable polyhydroxyalkanoates; and copolymers thereof.

Natural or naturally-based degradable polymers can include polysaccharides and modified polysaccharides such as starch, cellulose, chitin, chitosan, and copolymers thereof.

Specific examples of degradable polymers include poly(ether ester) multiblock copolymers based on poly(ethylene glycol) (PEG) and poly(butylene terephthalate) that can be described by the following general structure:

[—(OCH₂CH₂)ₙ—O—C(O)—C₆H₄—C(O)-]x[-O—(CH₂)₄—O—C(O)—C₆H₄—C(O)-]y,

where —C₆H₄— designates the divalent aromatic ring residue from each esterified molecule of terephthalic acid, n represents the number of ethylene oxide units in each hydrophilic PEG block, x represents the number of hydrophilic blocks in the copolymer, and y represents the number of hydrophobic blocks in the copolymer. n can be selected such that the molecular weight of the PEG block is between about 300 and about 4000. X and y can be selected so that the multiblock copolymer contains from about 55% up to about 80% PEG by weight. The block copolymer can be engineered to provide a wide array of physical characteristics (e.g., hydrophilicity, adherence, strength, malleability, degradability, durability, flexibility) and active agent release characteristics (e.g., through controlled polymer degradation and swelling) by varying the values of n, x and y in the copolymer structure.

Degradable polyesteramides can include those formed from the monomers OH-x-OH, z, and COOH-y-COOH, wherein x is alkyl, y is alkyl, and z is valine, leucine, isoleucine, norleucine, methionine, or phenylalanine.

Degradable polymeric materials can also be selected from: (a) non-peptide polyamino polymers; (b) polyiminocarbonates; (c) polycarbonates and polyarylates; and (d) poly(alkylene oxide) polymers.

Degradable polymers of the invention can also include polymerized polysaccharides such as those described in U.S. Pub. App. No. US 2005/0255142, entitled “COATINGS FOR MEDICAL ARTICLES INCLUDING NATURAL BIODEGRADABLE POLYSACCHARIDES”, U.S. Pub. App. No. 2007/0065481, entitled “COATINGS INCLUDING NATURAL BIODEGRADABLE POLYSACCHARIDES AND USES THEREOF”, and in U.S. Publ. App. No. 2007/0218102, entitled “BIODEGRADABLE HYDROPHOBIC POLYSACCHARIDE-BASED COATINGS”, all of which are herein incorporated by reference.

Degradable polymers of the invention can also include dextran based polymers such as those described in U.S. Pat. No. 6,303,148, entitled “PROCESS FOR THE PREPARATION OF A CONTROLLED RELEASE SYSTEM”. Exemplary dextran based degradable polymers including those available commercially under the trade name OCTODEX. Degradable polymers of the invention can further include collagen/hyaluronic acid polymers.

Various functional groups can be appended to degradable polymers in order to improve functional characteristics of the same. By way of example, in some embodiments, degradable polymers can include functional groups that increase the lubricity of the degradable pad in the presence of water, reducing the coefficient of friction of the degradable pad in vivo. Lubricity enhancing functional groups can specifically include functional groups that impart hydrophilic properties.

Polymeric layers used with embodiments of the invention can also include vapor and/or plasma deposited polymers. In an embodiment, the polymeric layer(s) include parylene and parylene derivatives. “Parylene” is both a generic name for a known group of polymers based on p-xylylene and made by vapor or plasma phase polymerization, and a name for the unsubstituted form of the polymer; the latter usage is employed herein for the term “parylene”. The term “parylene derivative” will refer to the known group of polymers based on p-xylylene and made by vapor or plasma phase polymerization. Common parylene derivatives include poly 2-chloro-paraxylylene (parylene C), polyparaxylylene (parylene N), and poly 2,5-dichloro-paraxylylene (parylene D). The polymeric layer can include mono-, di-, tri-, and tetra-halo substituted polyparaxylylene.

Parylene or a parylene derivative can be created by first heating p-xylene or a suitable derivative at an appropriate temperature (for example, at about 950° C.) to produce the cyclic dimer di-p-xylylene (or a derivative thereof). The resultant solid can be separated in pure form, and then cracked and pyrolyzed at an appropriate temperature (for example, at about 680° C.) to produce a monomer vapor of p-xylylene (or derivative); the monomer vapor is cooled to a suitable temperature (for example, below 50° C.) and allowed to condense on the desired object. An unsubstituted parylene polymer can have the repeating structure -(p-CH₂—C₆H₄—CH₂)ₙ—, with n equal to about 5,000 daltons, and a molecular weight of about 500,000 daltons. Parylene and parylene derivative coatings applicable by vapor deposition are commercially available from or through a variety of sources, including Specialty Coating Systems (100 Deposition Drive, Clear Lake, Wis. 54005), Para Tech Coating, Inc. (35 Argonaut, Aliso Viejo, Calif. 92656) and Advanced Surface Technology, Inc. (9 Linnel Circle, Billerica, Mass. 01821-3902).

The polymer layer(s) can be applied onto a porous reservoir body using various techniques such as dip-coating, spray-coating (including both gas-atomization and ultrasonic atomization), fogging, vapor deposition, brush coating, press coating, blade coating, and the like. The coating solutions can be applied under conditions where atmospheric characteristics such as relative humidity, temperature, gaseous composition, and the like are controlled.

Active Agents

Embodiments of medical devices and delivery systems described herein can elute or release one or more active agents. As used herein, the term “active agent” means a compound that has a particular desired activity. For example, an active agent can be a therapeutic compound that exerts a specific activity on a subject. In some embodiments, active agent will, in turn, refer to a peptide, protein, carbohydrate, nucleic acid, lipid, polysaccharide or combinations thereof, or synthetic inorganic or organic molecule, that causes a desired biological effect when administered in vivo to an animal, including but not limited to birds and mammals, including humans. Desired biological effects can include, but are not limited to, preventing or treating infection, modulating the immune response of the patient, modulating tissue growth, and the like. Active agents can include macromolecules, small molecules, hydrophilic molecules, hydrophobic molecules, and the like.

Active agents useful according to the invention include substances that possess desirable therapeutic characteristics for application to the implantation site. Active agents useful in the present invention can include many types of therapeutics including thrombin inhibitors, antithrombogenic agents, thrombolytic agents, fibrinolytic agents, anticoagulants, anti-platelet agents, vasospasm inhibitors, calcium channel blockers, steroids, vasodilators, anti-hypertensive agents, β-blockers, anti-anginal agents, cardiac inotropic agents, anti-arrhythmic agents, lipid regulating agents, antimicrobial agents, antibiotics, antibacterial agents, antiparasite and/or antiprotozoal agents, antiseptics, antifungals, antimalarials, angiogenic agents, anti-angiogenic agents, inhibitors of surface glycoprotein receptors, antimitotics, microtubule inhibitors, antisecretory agents, actin inhibitors, remodeling inhibitors, antisense nucleotides, anti-metabolites, miotic agents, anti-proliferatives, anticancer chemotherapeutic agents, anti-neoplastic agents, antipolymerases, antivirals, anti-inflammatory steroids or non-steroidal anti-inflammatory agents, analgesics, antipyretics, immunosuppressive agents, immunomodulators, growth hormone antagonists, growth factors, radiotherapeutic agents, peptides, proteins, enzymes, hormones, extracellular matrix components, ACE inhibitors, free radical scavengers, chelators, anti-oxidants, photodynamic therapy agents, gene therapy agents, anesthetics, opioids, dopamine agonists, antihistamines, tranquilizers, anticonvulsants, muscle relaxants, antispasmodics and muscle contractants, anticholinergics, ophthalmic agents, antiglaucoma solutes, prostaglandins, neurotransmitters, imaging agents, specific targeting agents, and cell response modifiers.

Active agents can specifically include anti-microbial agents such as antibiotics. Antibiotics are substances which inhibit the growth of or kill microorganisms. Antibiotics can be produced synthetically or by microorganisms. Examples of antibiotics include penicillin, tetracycline, tobramycin, chloramphenicol, minocycline, doxycycline, vancomycin, bacitracin, kanamycin, neomycin, polymyxin B, gentamycin, erythromycin, geldanamycin, geldanamycin analogs, cephalosporins, or the like. Examples of cephalosporins include cephalothin, cephapirin, cefazolin, cephalexin, cephradine, cefadroxil, cefamandole, cefoxitin, cefaclor, cefuroxime, cefonicid, ceforanide, cefotaxime, moxalactam, ceftizoxime, ceftriaxone, and cefoperazone.

Anti-microbial agents can specifically include anti-microbial peptides. Anti-microbial peptides can include those described in U.S. Pat. Nos. 5,945,507, 6,835,713, and 6,887,847, the contents of which are herein incorporated by reference.

Anti-microbial agents can also include antiseptics. Antiseptics are recognized as substances that prevent or arrest the growth or action of microorganisms, generally in a nonspecific fashion, e.g., either by inhibiting their activity or destroying them. Examples of antiseptics include silver sulfadiazine, chlorhexidine, glutaraldehyde, peracetic acid, sodium hypochlorite, triclosan, phenols, phenolic compounds, iodophor compounds, quaternary ammonium compounds, and chlorine compounds.

Active agents can specifically include antiviral agents. Antiviral agents are substances capable of destroying or suppressing the replication of viruses. Examples of anti-viral agents include α-methyl-1-adamantanemethylamine, hydroxy-ethoxymethylguanine, adamantanamine, 5-iodo-2′-deoxyuridine, trifluorothymidine, interferon, and adenine arabinoside.

Active agents can specifically include those agents capable of modulating bone and cartilage tissue growth. By way of example, active agents can include osteogenic growth peptide, insulin-like growth factor-1 (IGF-1), insulin, human growth hormone, activated vitamin D binding protein (ADBP), bone and cartilage stimulating peptide (such as BCSP-1), bone morphogenic proteins (including BMP-7), and platelet derived growth factor (PDGF). Other active agents capable of modulating bone and cartilage can specifically include peptides described in U.S. Pat. No. 5,635,482 (the contents of which is herein incorporated by reference) and commercially available under the trade name P-15.

Active agents can specifically include enzyme inhibitors. Enzyme inhibitors are substances that inhibit an enzymatic reaction. Examples of enzyme inhibitors include edrophonium chloride, N-methylphysostigmine, neostigmine bromide, physostigmine sulfate, tacrine HCL, tacrine, 1-hydroxy maleate, iodotubercidin, p-bromotetramisole, 10-(α-diethylaminopropionyl)-phenothiazine hydrochloride, calmidazolium chloride, hemicholinium-3,3,5-dinitrocatechol, diacylglycerol kinase inhibitor I, diacylglycerol kinase inhibitor II, 3-phenylpropargylaminie, N-monomethyl-L-arginine acetate, carbidopa, 3-hydroxybenzylhydrazine HCl, hydralazine HCl, clorgyline HCl, deprenyl HCl L(−), deprenyl HCl D(+), hydroxylamine HCl, iproniazid phosphate, 6-MeO-tetrahydro-9H-pyrido-indole, nialamide, pargyline HCl, quinacrine HCl, semicarbazide HCl, tranylcypromine HCl, N,N-diethylaminoethyl-2,2-di-phenylvalerate hydrochloride, 3-isobutyl-1-methylxanthne, papaverine HCl, indomethacind, 2-cyclooctyl-2-hydroxyethylamine hydrochloride, 2,3-dichloro-α-methylbenzylamine (DCMB), 8,9-dichloro-2,3,4,5-tetrahydro-1H-2-benzazepine hydrochloride, p-aminoglutethimide, p-aminoglutethimide tartrate R(+), p-aminoglutethimide tartrate S(−), 3-iodotyrosine, alpha-methyltyrosine L(−), alpha-methyltyrosine D(−), cetazolamide, dichlorphenamide, 6-hydroxy-2-benzothiazolesulfonamide, and allopurinol.

Active agents can specifically include anti-pyretics. Anti-pyretics are substances capable of relieving or reducing fever. Anti-inflammatory agents are substances capable of counteracting or suppressing inflammation. Examples of such agents include aspirin (salicylic acid), indomethacin, sodium indomethacin trihydrate, salicylamide, naproxen, colchicine, fenoprofen, sulindac, diflunisal, diclofenac, indoprofen and sodium salicylamide.

Active agents can specifically include anesthetics. Local anesthetics are substances that have an anesthetic effect in a localized region. Examples of such anesthetics include procaine, lidocaine, tetracaine and dibucaine.

Active agents can specifically include imaging agents. Imaging agents are agents capable of imaging a desired site, e.g., tumor, in vivo. Examples of imaging agents include substances having a label that is detectable in vivo, e.g., antibodies attached to fluorescent labels. The term antibody includes whole antibodies or fragments thereof.

Active agents can specifically include cell response modifiers. Cell response modifiers include chemotactic factors such as platelet-derived growth factor (PDGF). Other cell response modifiers can include neutrophil-activating protein, monocyte chemoattractant protein, macrophage-inflammatory protein, SIS (small inducible secreted), platelet factor, platelet basic protein, melanoma growth stimulating activity, epidermal growth factor, transforming growth factor alpha, fibroblast growth factor, platelet-derived endothelial cell growth factor, insulin-like growth factor, nerve growth factor, bone growth/cartilage-inducing factor (alpha and beta), and matrix metalloproteinase inhibitors. Other cell response modifiers include the interleukins, interleukin receptors, interleukin inhibitors, interferons, including alpha, beta, and gamma; hematopoietic factors, including erythropoietin, granulocyte colony stimulating factor, macrophage colony stimulating factor and granulocyte-macrophage colony stimulating factor; tumor necrosis factors, including alpha and beta; transforming growth factors (beta), including beta-1, beta-2, beta-3, inhibin, activin, and DNA that encodes for the production of any of these proteins, antisense molecules, androgenic receptor blockers and statin agents.

Other active agents can include heparin, covalent heparin, synthetic heparin salts, or another thrombin inhibitor; hirudin, hirulog, argatroban, D-phenylalanyl-L-poly-L-arginyl chloromethyl ketone, or another antithrombogenic agent; urokinase, streptokinase, a tissue plasminogen activator, or another thrombolytic agent; a fibrinolytic agent; a vasospasm inhibitor; a calcium channel blocker, a nitrate, nitric oxide, a nitric oxide promoter, nitric oxide donors, dipyridamole, or another vasodilator; HYTRIN® or other antihypertensive agents; a glycoprotein IIb/IIIa inhibitor (abciximab) or another inhibitor of surface glycoprotein receptors; aspirin, ticlopidine, clopidogrel or another antiplatelet agent; colchicine or another antimitotic, or another microtubule inhibitor; dimethyl sulfoxide (DMSO), a retinoid, or another antisecretory agent; cytochalasin or another actin inhibitor; cell cycle inhibitors; remodeling inhibitors; deoxyribonucleic acid, an antisense nucleotide, or another agent for molecular genetic intervention; methotrexate, or another antimetabolite or antiproliferative agent; tamoxifen citrate, TAXOL®, paclitaxel, or the derivatives thereof, rapamycin (or other rapalogs e.g. ABT-578 or sirolimus), vinblastine, vincristine, vinorelbine, etoposide, tenopiside, dactinomycin (actinomycin D), daunorubicin, doxorubicin, idarubicin, anthracyclines, mitoxantrone, bleomycin, plicamycin (mithramycin), mitomycin, mechlorethamine, cyclophosphamide and its analogs, chlorambucil, ethylenimines, methylmelamines, alkyl sulfonates (e.g., busulfan), nitrosoureas (carmustine, etc.), streptozocin, methotrexate (used with many indications), fluorouracil, floxuridine, cytarabine, mercaptopurine, thioguanine, pentostatin, 2-chlorodeoxyadenosine, cisplatin, carboplatin, procarbazine, hydroxyurea, morpholino phosphorodiamidate oligomer or other anti-cancer chemotherapeutic agents; cyclosporin, tacrolimus (FK-506), pimecrolimus, azathioprine, mycophenolate mofetil, mTOR inhibitors, or another immunosuppressive agent; cortisol, cortisone, dexamethasone, dexamethasone sodium phosphate, dexamethasone acetate, dexamethasone derivatives, betamethasone, fludrocortisone, prednisone, prednisolone, 6U-methylprednisolone, triamcinolone (e.g., triamcinolone acetonide), or another steroidal agent; trapidil (a PDGF antagonist), angiopeptin (a growth hormone antagonist), angiogenin, a growth factor (such as vascular endothelial growth factor (VEGF)), or an anti-growth factor antibody (e.g., ranibizumab, which is sold under the tradename LUCENTIS®), or another growth factor antagonist or agonist; dopamine, bromocriptine mesylate, pergolide mesylate, or another dopamine agonist; iodine-containing compounds, barium-containing compounds, gold, tantalum, platinum, tungsten or another heavy metal functioning as a radiopaque agent; a peptide, a protein, an extracellular matrix component, a cellular component or another biologic agent; captopril, enalapril or another angiotensin converting enzyme (ACE) inhibitor; angiotensin receptor blockers; enzyme inhibitors (including growth factor signal transduction kinase inhibitors); ascorbic acid, alpha tocopherol, superoxide dismutase, deferoxamine, a 21-aminosteroid (lasaroid) or another free radical scavenger, iron chelator or antioxidant; a ¹⁴C-, ³H-, ¹³¹I-, ³²P- or ³⁶ S-radiolabelled form or other radiolabelled form of any of the foregoing; an estrogen (such as estradiol, estriol, estrone, and the like) or another sex hormone; AZT or other antipolymerases; acyclovir, famciclovir, rimantadine hydrochloride, ganciclovir sodium, Norvir, Crixivan, or other antiviral agents; 5-aminolevulinic acid, meta-tetrahydroxyphenylchlorin, hexadecafluorozinc phthalocyanine, tetramethyl hematoporphyrin, rhodamine 123 or other photodynamic therapy agents; an IgG2 Kappa antibody against Pseudomonas aeruginosa exotoxin A and reactive with A431 epidermoid carcinoma cells, monoclonal antibody against the noradrenergic enzyme dopamine beta-hydroxylase conjugated to saporin, or other antibody targeted therapy agents; gene therapy agents; enalapril and other prodrugs; PROSCARR®, HYTRINR® or other agents for treating benign prostatic hyperplasia (BHP); mitotane, aminoglutethimide, breveldin, acetaminophen, etodalac, tolmetin, ketorolac, ibuprofen and derivatives, mefenamic acid, meclofenamic acid, piroxicam, tenoxicam, phenylbutazone, oxyphenbutazone, nabumetone, auranofin, aurothioglucose, gold sodium thiomalate, a mixture of any of these, or derivatives of any of these. Active agents can specifically include microparticles. For example, active agents, such as those described above, can be formulated as microparticles and disposed within interconnected pores.

It will be understood that changes and modifications may be made without departing from the scope and the spirit of the invention as hereinafter claimed. The invention will now be demonstrated referring to the following non-limiting examples.

### EXAMPLES

### Example 1

### Elution of Active Agent from Porous Ceramic Article with Parylene Top Coat

An active agent solution was prepared by dissolving 500 mg of tobramycin in 1 milliliter of water. Porous ceramic (alumina) disks (n=2) were obtained from Small Parts, Inc. (Miami Lakes, Fla.). The porous ceramic disks had a diameter of 16 millimeters and a height of 7 millimeter (total volume≈1407 mm³). The pores of the ceramic disks formed an open cell network with a void volume equal to 34% of the total volume of the disk (void volume≈478 mm³). Roughly 500 microliters of the active agent solution was pipetted onto each of the porous ceramic disks. The active agent solution was allowed to soak in and then was dried over a period of roughly 48 hours.

A first coating solution was prepared by dissolving PBMA in chloroform to a concentration of 100 mg/milliliter. Each of the ceramic disks were then dipped into the first coating solution for a period of approximately 1 minute and then removed and allowed to dry.

One of the disks (disk #1) was then coated with a layer of parylene-C. Specifically, 2 grams of Parylene C dimer (Specialty Coating Systems, Indianapolis, Ind.) was loaded into a vapor deposition system PDS-2010 LABCOTER® (Specialty Coating Systems, Indianapolis, Ind.). A coating cycle was then initiated and a layer of Parylene approximately 1 to 2 microns thick was deposited onto disk #1 under vacuum.

A second coating solution was prepared by dissolving PBMA in isopropyl alcohol to a concentration of 50 mg/ml. One of the porous ceramic disks (disk #2) was dipped into the second coating solution for a period of approximately 1 minute and then removed and allowed to dry.

Elution of tobramycin from the ceramic disks was then tested. Elution of tobramycin was carried out in 20 mL PBS, pH 7.4, at 37° C. Samples were shaken gently for the duration of the experiment. The buffer solution was refreshed after each elution measurement. The amount of tobramycin eluted during a particular time increment was quantified with the fluorescent tag fluorescamine (TCI America, Portland, Oreg.), which fluoresces only after reacting with free amines such as those presented by tobramycin. 90 μL of eluent was removed from each sample vial and placed into a black 96-well plate. PBS blanks and standard solutions (tobramycin concentrations between 1 and 1000 μg/mL in PBS) were placed on the same plate. 6 μL of fluorescamine solution (10 mg/mL in acetone) was added to each well and the plate was read on a SpectraMax Gemini spectrophotometer (Molecular Devices, Sunnyvale, Calif.). The excitation and emission wavelengths were 400 and 460 nm, respectively. Serial dilutions (10×) were performed as necessary to ensure that the sample fluorescent intensity corresponded to the range of the standard curve. The amount of tobramycin present in solution and the total amount of tobramycin eluted for each time increment was calculated from the standard curve.

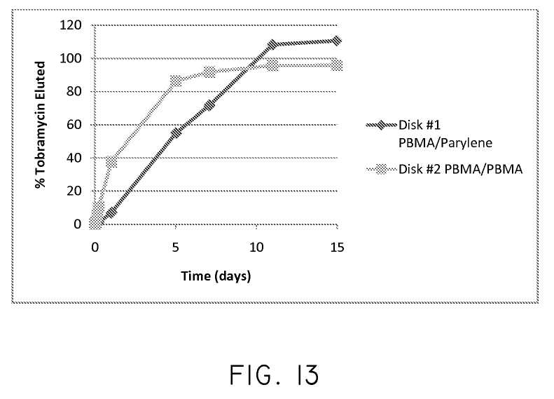

The elution results are shown below in Table 1 and in FIG. 13. The data show that a porous disk with a parylene topcoat was able to elute tobramycin with near zero-order elution kinetics.

TABLE 1

% Tobramycin Eluted

| Time | Disk #1 | Disk #2 |
|---|---|---|
| (days) | PBMA/Parylene | PBMA/PBMA |
| 0.000 | 0.00 | 0.00 |
| 0.041 | 0.14 | 1.17 |
| 0.125 | 0.26 | 5.33 |
| 0.229 | 1.00 | 10.19 |
| 1.021 | 7.06 | 37.62 |
| 5.021 | 55.01 | 86.31 |
| 7.083 | 71.75 | 91.86 |
| 11.000 | 108.37 | 95.71 |
| 14.958 | 110.73 | 95.87 |

### Example 2

### Effect of Parylene Layer Thickness on Elution of Active Agent from Porous Ceramic Article

An active agent solution was prepared by dissolving 500 mg of tobramycin in 1 milliliter of water. Porous ceramic (alumina) disks (n=3) were obtained from Small Parts Inc. (Miami Lakes, Fla.). The porous ceramic disks had a diameter of 16 millimeters and a height of 7 millimeters (total volume≈1407 mm³). The pores of the ceramic disks formed an open cell network with a void volume equal to 34% of the total volume of the disk (void volume≈478 mm³). Each disk was weighed as reflected in Table 2 below.

Roughly 450-475 microliters of the active agent solution was pipetted onto each of the porous ceramic disks, allowed to soak for one hour and dried under vacuum overnight. The disks were then flipped over and an additional 200 microliters of the active agent was pipette onto the disks and allowed to soak in. Finally, the disks were flipped over one final time and an additional 100 microliters of the active agent solution was pipetted onto the disk surface. This final 100 microliters was observed not to soak in, even after three hours. The disks were then dried under vacuum over a period of roughly 48 hours. Each disk was then weighed again. The weights of the disks before and after addition of the active agent are shown below in Table 2.

TABLE 2

| | Weight (mg) | | |
|---|---|---|---|
| | | Disk + | |
| | | Active | Active |
| Disk # | Bare Disk | Agent | Agent |
| 3 | 2565.8 | 2964.8 | 399 |
| 4 | 2546 | 2925.3 | 379.3 |
| 5 | 2574.8 | 2971.6 | 396.8 |

A coating solution was prepared by dissolving PBMA in chloroform to a concentration of 200 mg/milliliter. Each of the ceramic disks were then dipped into the coating solution twice for a period of approximately 1 minute each time and then removed and allowed to dry.

Each of the disks were then coated with a layer of parylene-C. Varying amounts of Parylene C dimer (Specialty Coating Systems, Indianapolis, Ind.) was loaded into a vapor deposition system PDS-2010 LABCOTER® (Specialty Coating Systems, Indianapolis, Ind.) for each disk. Specifically, for disk #3, three grams of Parylene C dimer was used. For disk #4, six grams of Parylene C dimer was used. For disk #5, nine grams of Parylene C dimer was used. A coating cycle was then initiated and a layer of parylene was deposited onto the disks. The parylene layer was approximately 1 to 2 microns thick for disk #3, 2 to 4 microns thick for disk #4, and 4 to 6 microns thick for disk #5.

Elution of tobramycin from the ceramic disks was then tested according to the procedure described above in Example 1.

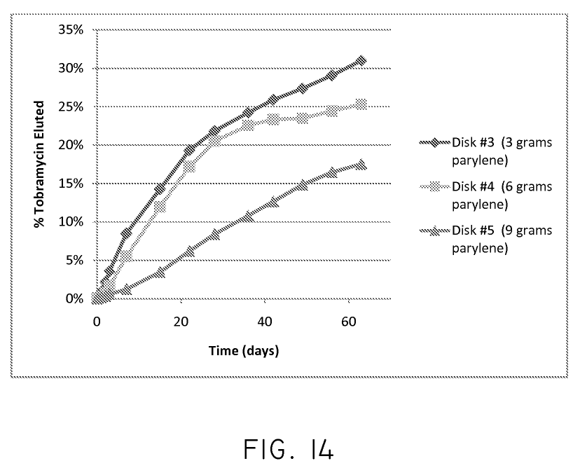

The elution results are shown below in Table 3 and in FIG. 14. The data show that increasing amounts of parylene resulted in slower elution kinetics. The data also show that porous disks with parylene topcoats are able to elute tobramycin with near zero-order elution kinetics. In addition, the data show that this coating configuration can be used to achieve near zero-order release kinetics over a period of time exceeding 60 days.

TABLE 3

| | % Tobramycin Eluted | | |
|---|---|---|---|
| | Disk #3 | Disk #4 | Disk #5 |
| Time | (3 grams | (6 grams | (9 grams |
| (days) | parylene) | parylene) | parylene) |
| 0.000 | 0.0% | 0.0% | 0.0% |
| 0.042 | 0.0% | 0.0% | 0.0% |
| 0.125 | 0.1% | 0.1% | 0.1% |
| 0.250 | 0.2% | 0.1% | 0.1% |
| 1.000 | 0.91% | 0.25% | 0.22% |
| 2.000 | 2.18% | 0.70% | 0.36% |
| 2.958 | 3.56% | 1.64% | 0.59% |
| 7.000 | 8.49% | 5.52% | 1.24% |
| 15.000 | 14.3% | 12.0% | 3.5% |
| 22.000 | 19.32% | 17.23% | 6.20% |
| 28.000 | 21.83% | 20.51% | 8.40% |
| 36.000 | 24.22% | 22.56% | 10.81% |
| 42.000 | 25.88% | 23.36% | 12.66% |
| 49.000 | 27.37% | 23.48% | 14.85% |
| 56.000 | 29.07% | 24.44% | 16.46% |
| 63.000 | 31.00% | 25.32% | 17.54% |

It should be noted that, as used in this specification and the appended claims, the singular forms “a”, “an”, and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration to. The phrase “configured” can be used interchangeably with other similar phrases such as arranged and configured, constructed and arranged, adapted, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

The invention has been described with reference to various specific embodiments and techniques. However, it should be understood that many variations and modifications may be made while remaining within the spirit and scope of the invention.

### Further Embodiments

In an embodiment, the invention includes an active agent delivery system including a reservoir body defining a plurality of interconnected pores, an active agent disposed within the interconnected pores, and a first polymeric layer disposed over the reservoir body. In an embodiment, the first polymeric layer includes a parylene. In an embodiment, the first polymeric layer includes a poly-alkyl-methacrylate. In an embodiment, the first polymeric layer includes poly-n-butyl-methacrylate (PBMA), polyethylene-co-vinyl-acetate (PEVA), or a combination of PBMA and PEVA. In an embodiment, the first polymer layer includes poly-n-butyl-methacrylate. In an embodiment, the first polymer layer has a thickness of about 0.1 micrometers to about 100 micrometers. In some embodiment, the active agent delivery system can include a second polymer layer disposed over the first polymer layer, the first polymer layer including a poly-alkyl-methacrylate and the second polymer layer comprising a parylene. In some embodiments, the interconnected pores can include a polar surface. In some embodiments, the interconnected pores can include a negatively charged surface. In some embodiments, the interconnected pores can include a positively charged surface. In some embodiments, the interconnected pores can include a non-polar surface. In some embodiments, the reservoir body can include a ceramic. In some embodiments, the ceramic can be selected from the group consisting of alumina, hydroxyapatite, calcium phosphate, pyrolytic carbon, sapphire, silica, silicon carbide, silicon nitride, zirconia. In an embodiment, the reservoir body includes a metal. In an embodiment, the metal can be selected from the group consisting of titanium, titanium alloys, iron-chrome-nickel alloys, and cobalt-chrome alloys. In some embodiments, the reservoir body can have structural rigidity. In some embodiments, the reservoir body comprising a material having a shear modulus of greater than about 3 GPa. In some embodiments, the reservoir body can include a material having a Rockwell hardness of greater than about HRC 40. In some embodiments, the reservoir body can comprise a polymer. In some embodiments, the polymer can have a Shore durometer hardness of at least about 50D. In some embodiments, the reservoir body can include a polymer selected from the group consisting of polyethylenes, polysiloxanes, polypropylenes, and polyamides. In some embodiments, the interconnected pores can include an average diameter of between 0.1 micrometers and 50 micrometers. In some embodiments, the interconnected pores comprising an average diameter of between 0.5 micrometers and 20 micrometers. In some embodiments, the active agent can include a polar active agent. In some embodiments, the active agent can include a positively charged active agent. In some embodiments, the active agent can have anti-microbial activity. In some embodiments, the active agent can include an antibiotic. In some embodiments, the active agent can include one or more of tobramycin, vancomycin, and penicillin G. In some embodiments, the active agent can include tobramycin. In some embodiments, the active agent can include an agent capable of modulating bone and cartilage tissue growth. In some embodiments, the active agent can include a non-polar active agent. In some embodiments, the plurality of interconnected pores can include a first interconnecting network of pores and a second interconnecting network of pores, the active agent disposed within the first interconnecting network of pores and a second active agent disposed within the second interconnecting network of pores. In an embodiment, the system can be configured to elute the active agent with zero-order kinetics. In some embodiments, the system can be configured to elute an amount of the active agent between 30 and 60 days that is at least equal to 90% of the amount eluted between 0 days and 30 days. In some embodiments, the system can be configured to elute at least about 20% of the total amount of the active agent after being disposed in vivo for at least 60 days. In some embodiments, the reservoir body can have a porosity of about 5% to about 90%. In some embodiments, the reservoir body can have a porosity of about 30% to about 50%. In some embodiments, the active agent delivery system can also include a non-porous support layer disposed under the reservoir body. In some embodiments, the active agent delivery system can also include a second reservoir body defining a second plurality of interconnected pores.

In an embodiment, the invention can include an implantable medical device including a porous substrate defining a plurality of interconnected pores, an active agent disposed within the interconnected pores, and a first polymeric layer disposed over the reservoir body. In an embodiment, the porous substrate can have a spherical shape. In an embodiment, the porous substrate can include a first bead. In an embodiment, the device can include a second bead, the second bead comprising a second porous substrate defining a second plurality of interconnected pores, a second active agent disposed within the second plurality of interconnected pores, and a second polymeric layer disposed over the second porous substrate. In an embodiment, the first polymeric layer can encapsulate the reservoir body.

In an embodiment, the invention can include a method of making an active agent delivery system. The method can include forming a porous reservoir body, inserting an active agent within the porous reservoir body, and applying a polymeric layer over the porous reservoir body. In an embodiment, forming a porous reservoir body can include performing a phase extraction operation. In an embodiment, inserting an active agent within the porous reservoir body can include dissolving the active agent in a solvent to form an active agent solution and then applying the active agent solution to the porous reservoir body. In an embodiment, applying a polymeric layer over the porous reservoir body can include performing a dip coating operation. In an embodiment, applying a polymeric layer over the porous reservoir body can include performing a dip coating operation. In an embodiment, applying a polymeric layer over the porous reservoir body can include performing a spray coating operation. In an embodiment, applying a polymeric layer over the porous reservoir body can include performing a vapor deposition operation.

1. An active agent delivery system comprising:
a reservoir body defining a plurality of interconnected pores; the reservoir body comprising a material selected from the group consisting of ceramics and metals;
an active agent disposed within the interconnected pores; and
a first polymeric layer disposed over the reservoir body.

2. The active agent delivery system of claim 1, the first polymeric layer comprising a parylene.

3. The active agent delivery system of claim 1, the first polymeric layer comprising a poly-alkyl-methacrylate.

4. The active agent delivery system of claim 1, the first polymeric layer comprising poly-n-butyl-methacrylate (PBMA), polyethylene-co-vinyl-acetate (PEVA), or a combination of PBMA and PEVA

5. The active agent delivery system of claim 1, further comprising a second polymer layer disposed over the first polymer layer, the first polymer layer comprising a poly-alkyl-methacrylate and the second polymer layer comprising a parylene.

6. The active agent delivery system of claim 1, the interconnected pores comprising a polar surface.

7. The active agent delivery system of claim 1, the interconnected pores comprising a non-polar surface.

8. The active agent delivery system of claim 1, the interconnected pores comprising an average diameter of between 0.1 micrometers and 50 micrometers.

9. The active agent delivery system of claim 1, the reservoir body comprising a ceramic.

10. The active agent delivery system of claim 9, the ceramic selected from the group consisting of alumina, hydroxyapatite, calcium phosphate, pyrolytic carbon, sapphire, silica, silicon carbide, silicon nitride, zirconia.

11. The active agent delivery system of claim 1, the reservoir body comprising a metal.

12. The active agent delivery system of claim 11, the metal selected from the group consisting of titanium, titanium alloys, iron-chrome-nickel alloys, and cobalt-chrome alloys.

13. The active agent delivery system of claim 1, the system configured to elute an amount of the active agent between 30 and 60 days that is at least equal to 90% of the amount eluted between 0 days and 30 days.

14. The active agent delivery system of claim 1, the system configured to elute at least about 20% of the total amount of the active agent after being disposed in vivo for at least 60 days.

15. The active agent delivery system of claim 1, further comprising a second reservoir body defining a second plurality of interconnected pores.

16. An active agent delivery system comprising:
a reservoir body defining a plurality of interconnected pores; the reservoir body comprising a polymer, the polymer selected from the group consisting of polyethylenes, polysiloxanes, polyurethanes, polypropylenes, polyethers, polyesters, and polyamides;
an active agent disposed within the interconnected pores; and
a first polymeric layer disposed over the reservoir body.

17. The active agent delivery system of claim 16, the polymer having a Shore durometer hardness of at least about 50D.

18. The active agent delivery system of claim 16, the active agent comprising tobramycin.

19. The active agent delivery system of claim 16, the reservoir body being substantially flexible.

20. A method of making an active agent delivery system comprising:
forming a porous reservoir body;
inserting an active agent within the porous reservoir body; and
applying a polymeric layer over the porous reservoir body.
